(19) United States
(12) Patent Application Publication
(10) Pub. No.: US 2008/0075794 A1
(43) Pub. Date: Mar. 27, 2008

(54) Natural antimicrobial and method of manufacturing the same

(76) Inventor: ChangSoon Bae, Kwacheon-si (KR)

Correspondence Address: THE FARRELL LAW FIRM, P.C., 333 EARLE OVINGTON BOULEVARD, SUITE 701, UNIONDALE, NY, 11553, US

(21) Appl. No.: 11/881,843

(22) Filed: Jul. 30, 2007

(30) Foreign Application Priority Data
Aug. 1, 2006 (KR) 10-2006-0072455

(51) Int. Cl. A61K 36/00; A61P 43/00

(52) U.S. Cl. 424/725

(57) ABSTRACT

Disclosed is a method of and apparatus for manufacture of a natural antimicrobial, in which a first mixed solution is created by mixing Taxus extract, Thymus extract and ethanol; a second mixed solution is created by mixing oleic acid with the first mixed solution; a third mixed solution is created by mixing phytoncide with the second mixed solution; and a fourth mixed solution is created by mixing polysorbate with the third mixed solution, and the natural antimicrobial is completed by mixing ethanol, emulsification and water with the fourth mixed solution.

### PRIORITY

This application claims priority under 35 U.S.C. §119(a) to an application filed in the Korean Industrial Property Office on Aug. 1, 2006 and assigned Patent Application Serial No. 10-2006-0072455, the contents of which are hereby incorporated by reference.

### FIELD OF INVENTION

The current invention is related to a natural antimicrobial, and its method of manufacturing, that is capable of strong antimicrobial ability as well as the elimination of foul odor by the use of extracts from natural plants.

### BACKGROUND OF THE INVENTION

Generally, when microorganisms or germs ingest and break down sweat, sebum, or contaminants secreted by human body, it causes inflammation due to strong stimulus to the skin, and bad odor can generate due to Staphylococcus Aureus, Escherichia Coli, and other germs.

Especially in the case of human body's armpits, feet, head, and genital organs, because the humidity and temperature conditions are suitable and there are plenty of nutrients for germs to consume, they provide microorganisms such as bacteria, fungi, pathogenic bacteria, germs that cause athlete's foot, and mold with optimal conditions for survival and propagation, causing stimulus to the skin, itching, inflammation and blisters, rash, and bad odors created by the various inhabiting microorganisms.

Also, if sweat, sebum or other secretions secreted by the human body gets on objects such as clothing, furniture, and shoes, it could cause intense foul odor from various microorganisms prosperously propagating, and because it is difficult to get rid of all of these harmful germs even with frequent cleaning of these clothing, furniture, shoes, etc, the increased exposure to these harmful microorganisms during group life such as army base camps, dormitory, camps is the cause of serious infections and foul odors.

Recently, to solve these problems, various antimicrobials that control the propagation of various microorganisms and get rid of bad odor caused by these microorganisms have been developed. Also developed and widely used are mold remover, chemical detergents that kills various germs on clothing and furniture, skin medicine that exterminates germs that inhabit the human body and provide the cause for inflammation, etc.

However, because different types of antimicrobials are needed for different microorganisms, and because different types of antimicrobials are needed depending on the places, objects, and human body that need germs to be eliminated, it is difficult for consumers to make the right distinctions, and the consumers' expenses also rise.

Therefore, an antimicrobial that has a wide scope of uses is urgently demanded.

In addition, the recently developed and used antimicrobial is composed mostly of chemically processed materials that can cause environmental pollution or cause side effects that are harmful to the human body due to non-environment-friendly components.

### SUMMARY OF THE INVENTION

Accordingly, the current invention is developed to solve the problems listed above, and the object is to provide a natural antimicrobial and its method of manufacturing that can provide strong antimicrobial abilities, eliminate foul odors and various spawns as well as microorganism, creating germs while being harmless to the human body, by using the extracts from processed natural plant foodstuff that comes from Taxus as well as Thymus.

According to the natural antimicrobial according to the current invention, to accomplish the above stated objects, natural antimicrobial that includes Taxus extract extracted from Taxus and Thymus extract extracted from Thymus is provided.

According to the natural antimicrobial manufacturing method according to the current invention, to accomplish the above stated objects, the natural antimicrobial manufacturing method, characterized by including a step in which the 1^{st} mixed solution is created by mixing the above extracts from Taxus and Thymus with ethanol, a step in which the 2^{nd} mixed solution is created by mixing the above 1^{st} mixed solution with oleic acid, a step in which the 3^{rd} mixed solution is created by mixing the above 2^{nd} mixed solution with phytoncide, a step in which the 4^{th} mixed solution is created by mixing the above 3^{rd} mixed solution with polysorbate, a step in which the natural antimicrobial is created by mixing the above 4^{th} mixed solution, ethanol, emulsification, and water, is provided.

### BRIEF DESCRIPTION OF THE DRAWINGS

The above and other aspects, features and advantages of the present invention will be more apparent from the following detailed description taken in conjunction with the accompanying drawings, in which:

FIG. 1 is a flowchart that explains the manufacturing processes of the Taxus extract, which is a composition of the natural antimicrobial according to the current invention;

FIG. 2 is a flowchart that explains the manufacturing processes of the Thymus extract, which is a composition the natural antimicrobial according to the current invention;

FIG. 3 is a flowchart that explains the manufacturing processes of the natural antimicrobial using the Taxus extract and the Thymus extract according to the current invention;

From FIG. 4a to FIG. 4p, is an exemplifying photograph of the result of an experiment in which the natural antimicrobial was used to disinfect various strains of germs;

FIG. 5a and FIG. 5b, is an exemplifying photograph of a part of the human skin that shows the change in the condition of the skin over time after the natural antimicrobial was sprayed onto it;

From FIG. 6a to FIG. 6i, is an exemplifying photograph that shows the healing process after the natural antimicrobial was sprayed onto a diseased part of the human skin; and

FIGS. 7a and 7b, is an exemplifying photograph that shows the condition changes of bread that has been sprayed with the natural antimicrobial and bread that has not been sprayed with the antimicrobial.

### DETAILED DESCRIPTION OF THE PREFERRED EMBODIMENTS

The natural antimicrobial's composition according to the current invention uses extracts extracted from natural plants, and Taxus and Thymus is applied as these natural plants.

The Taxus stated above is an acicular tree that belong to Taxaceae, inhabiting not only the Asian continent's Temperate and Frigid Zones including all areas of South Korea, Japan, Manchuria, and Siberia, but also spreading over Europe as well as North America, it can grow up to 17 m in height and 1 m in diameter, and because it grows at a very slow rate of 1 mm diameter per year, the wood's constitution is very precise, and it does not decay readily when dead.

The bark of the stem of the Taxus stated above is reddish brown in color, and because it reveals glossy red color when the bark is removed it is called Joo Mok, or Red Tree, in Korean. Since the past, this tree has been used as civilian medicinal stuff to treat nephritis, edema, diabetes, etc, and currently is being studied for its potential use in anticancer medicines.

Also, the Thymus (scientific name: Thymus Quinquecostatus) stated above is distributed widely in the Asian continent including Korea, Japan, China, India, etc, along mountain summits, the hills, and between the rocks of seashores, and it is called Baek li hyang (Japonica) in Korean because its flowers' scent is very strong.

The Thymus stated above has a height between 3 and 15 cm, the stem is spread over the ground and the young scented branches stand slanted, and the leaves grow facing each other in an oval shape with the length 3-12 mm and the width 3-8 mm, has pellucid dots on both sides, and has plain and smooth edges, but has wave-shaped teeth and hair.

The whole plant of this Thymus is 1-2% of essential oil, which its content is the highest when the flowers are blooming, and 10-30% of the substance found in essential oil contains phenol substances. Essential oil's main ingredient is Thymol, which has less poisonous than other phenol substances, has week stimulating effect on the mucous membrane, and has strong antimicrobial and germ suppressing effect on pathogenic bacteria and molds.

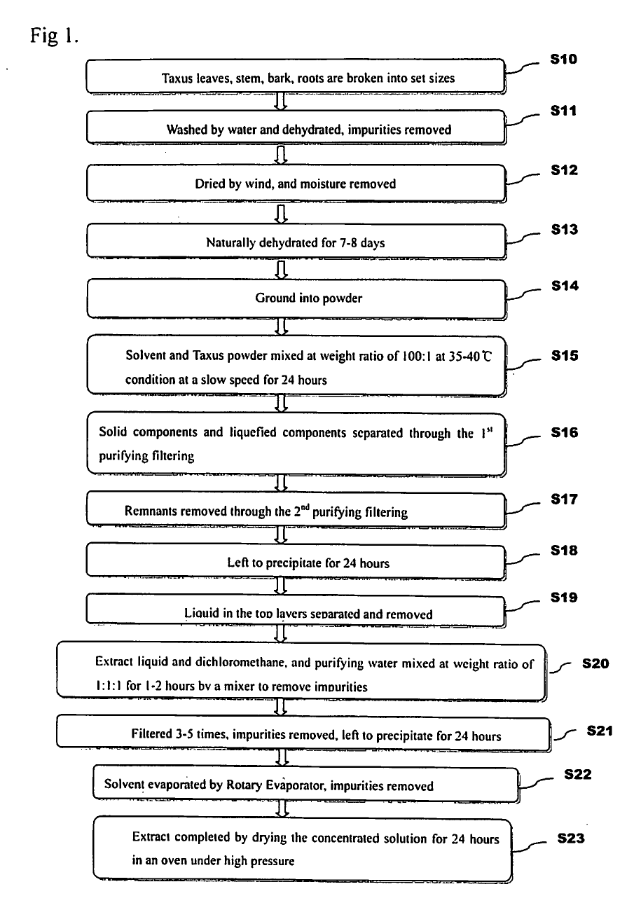

Explained next is the process in which Taxus extract is manufactured with reference to FIG. 1.

In other words, FIG. 1 is a flowchart that explains the process in which Taxus extracts that is included in the composition of the natural antimicrobial is manufactured.

First, the leaves, stem, bark, and the roots of Taxus that make up the composition of the current invention's natural antimicrobial are broken apart into about 0.5-1 cm size (step S10). The broken apart pieces are then washed with water then dehydrated, then they go through the process of eliminating impurities such as dust and impurities, etc, that may have been stuck on the Taxus (step S11).

After that, the pieces that have been washed and dehydrated, then was rid of impurities as described above go through the process in which the remaining moisture from the above wash with water is eliminated by drying with wind of temperature 25°-30° (Step S12). Then, these pieces that have been rid of moisture is dried for 7-8 days in natural conditions (step S13).

The naturally dried Taxus pieces described above are then ground into powder form of 50-300 Mesh particle size using a grinder (step S14). The ground Taxus powder is then mixed with a solvent (i.e. Ethanol) in 1:100 weight ratio (in other words, powder 1 g per solvent 100 g) and is mixed at 100-300 RPM speed by a mixer for 24 hours in 35°-40° temperature condition (step S15).

Mixed substance of Taxus powder and solvent as described above goes through the 1^{st} Purifying Filtering Process in which the liquefied layer is put through a filter paper or a filter in a funnel to separate liquid component from solid particles (step S16), and then only the liquid component is taken for the 2^{nd} Purifying Filtering Process in which the liquid component is put through a filter paper or a filter in a funnel to eliminate the remaining remnants (step S17).

The filtered substance that has been through the 2^{nd} Purifying Filtering Process is then left alone for 24 hours for precipitation, then only the supernatant from the substance that went through the precipitation process is separated (step S19).

After that, to eliminate impurities found in the extract, which is the separated supernatant, the extract, dichloromethane, and purifying water is combined in 1:1:1 weight ratio and mixed by a mixer for 1-2 hours (step S21).

The mixed substance of the extract, dichloromethane, and purifying water as described above goes through the filtering process, in which they are put through a funnel, repeatedly 3-5 times to eliminate impurities, and then is left alone for precipitation for 24 hours (step S21).

After going through the precipitation process described above, the substance is put into a Rotary Evaporator to evaporate the solvent (i.e. ethanol, dichloromethane, purifying water) included in the Taxus extract as well as to eliminate impurities (step S22).

After that, the final Taxus extract is completed by drying the concentrated solution concentrated by the Rotary Evaporator in an oven at 50°-100°, 1 steam pressure for 24 hours.

The main components of the completed Taxus extract as described above are 12.87-15.73 weight % of Alkaloid Taxine, 5.39-6.58 weight % of Ponasterone A, 3.90-4.78 weight % of Sciadopitysin, 12.33-15.07 weight % of Taxusin, 11.04-16.56 weight % of Taxinine A, B, H, K, L, 4.04-6.06 weight % of Ecdysterone, 38.03-46.47 weight % of taxol, 0.70-1.06 weight % of other substance.

On the other hand, the ideal weight ratio of the main components of Taxus extract is Taxine 14.3 weight %, Ponasterone A 5.98 weight %, Sciadopitysin 4.34 weight %, Taxusin 13.7 weight %, Taxinine A, B, H, K, L 13.8 weight %, Ecdysterone 5.05 weight %, Taxol 42.25 weight %, other substance 0.88 weight %.

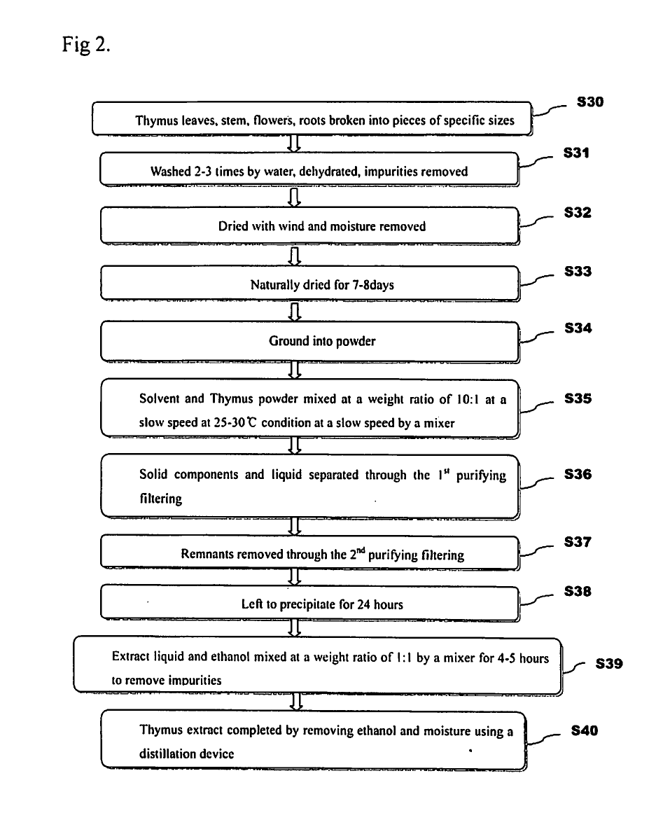

Next explained is the process in which Thymus extract that is included in the composition of the current invention's natural antimicrobial is manufactured with reference to FIG. 2.

In other words, FIG. 2 is a flowchart that explains the process in which Thymus extracts that is included in the composition of the natural antimicrobial is manufactured.

First, the Thymus leaves, stem, flowers, and roots are broken apart into pieces about 0.5-1 cm in size (step S30), then these pieces are washed with water 2-3 times, dehydrated, then removed of impurities (step S31).

The Thymus pieces that has been washed, dehydrated, and removed of impurities as described above is then removed of remaining moisture from the water used in washing described above by drying with wind at normal temperature (step S32), and dried for 7-8 days in natural conditions (step S33).

The Thymus piece that has been naturally dried as described above is then ground into powder form of 50-300 Mesh particle size by a grinder (step S34). The ground thymus powder is mixed with a solvent (i.e. 99.99% Pure Purifying water at 1:10 weight ratio (in other words, thymus powder 1 g per purifying water 10 g) and mixed for 24 hours at 100-300 RPM speed by a mixer at about 25°-30° temperature condition (step S35).

The mixed solution of thymus powder and solvent as described above goes through the 1^{st} Purifying Filtering Process in which the liquefied layer is put through a filter paper or a filter in a funnel to separate the liquid component from the solid component (step S36), and then only the liquid component goes through the 2^{nd} Filtering Process in which the liquid component is put through a filter paper or a filter in a funnel to remove the remaining remnants (step S37).

The filtered substance that has been through the 2^{nd} Purifying Filtering Process is then left alone for 24 hours for precipitation (step S38), and then, to remove impurities from the extract that has been removed of precipitated remnants during the precipitation process described above, the extract is combined with ethanol at 1:1 weight ratio, mixed for 4-5 hours, and purified, removing the impurities (step S39).

When the removal of impurities from the mixed solution of extracts and ethanol as described above is completed, distillation device is used to extract only the extracts and to complete the final thymus extract by removing ethanol and other moistures.

The main components of the completed thymus extract as described above include Thymol 31.31-38.39 weight %, Carvacrol 4.77-5.83 weight %, γ-Terpinene 7.2-8.8 weight % Borneol 4/5-5.5 weight %, Ketoconazole 30.18-36.88 weight %, P-Cymeme 1.36-2.04 weight %, \acute{α}(alpha)-Terpineol 4.0-6.0 weight %, \acute{α}(alpha)-Pinene 4.55-6.83 weight %, other substance 0.46-0.7 weight %.

On the other hand, the ideal weight ratio of the main components of the thymus extract as described above is Thymol 34.9 weight %, Carvacrol 5.3 weight %, γ-Terpinene 8.0 weight %, Borneol 5.0 weight %, Ketoconazole 33.53 weight %, P-Cymeme 1.7 weight %, \acute{α}(alpha)-Terpineol 5.0 weight %, \acute{α}(alpha)-Pinene 5.69 weight %, other substance 0.58 weight %.

According to the current invention, substance extracted from Taxus as described above (from now on “Taxus Extract”) and substance extracted from Thymus as described above (from now on “Thymus Extract”) are used to create the natural antimicrobial, and the substances to be mixed with this natural antimicrobial are Taxus extract, Thymus extract, oleic acid (Oliver), polysorbate, phytoncide, Ethanol, deionized water, and emulsification.

Taxus extract as described above is an antioxidant and has excellent antimicrobial capabilities against pathogenic germs and bacteria, while thymus extract as described above has excellent antimicrobial capabilities against fungi.

Oleic acid listed above has melting point of 12° and boiling point of 360° and is an unsaturated fatty acid with one double bond, which almost never melts in water but melts in ethanol, ether, and chloroform, and is included in vegetable oils such as olive oil and camellia oil. This oleic acid protects the human skin, prevents tolerance to antimicrobials and is also used in increasing immunity to virus.

Polysorbate listed above is an activator high in hydrophilic properties and is a non-polar emulsifier used as an emulsifying agent in food and medical supplies. phytoncide listed above is a substance included in almost every tree which effects the growth and propagation of microorganisms, preventing oxidation and eliminating odor.

Emulsification listed above is a liquid disperse system with particle size of 20-200 nm, being used as medicine delivery form because of its skin-like liquid crystal interface membrane that has small particles, and having a tendency to stick to microorganisms only, and not to human body cells, due to emulsification's special surface charge.

The composition ratio of the each substance mixing in the Natural antimicrobial is as shown in the Table 1 below.

TABLE 1

| | | |
|---|---|---|
| Composition | Weight Ratio | Ideal Weight |
| Substance | Range | Ratio |

| | | | | |
|---|---|---|---|---|
| Taxus Extract | 2-7 | Weight % | 5 | Weight % |
| Thymus Extract | 2-7 | Weight % | 5 | Weight % |
| Oleic Acid | 2-5 | Weight % | 3.75 | Weight % |
| polysorbate | 0.1-1.0 | Weight % | 0.5 | Weight % |
| phytoncide | 0.1-1.0 | Weight % | 0.5 | Weight % |
| Ethanol | 3-8 | Weight % | 5 | Weight % |
| Water | 75-80 | Weight % | 78.25 | Weight % |
| Emulsification | 1-3 | Weight % | 2 | Weight % |

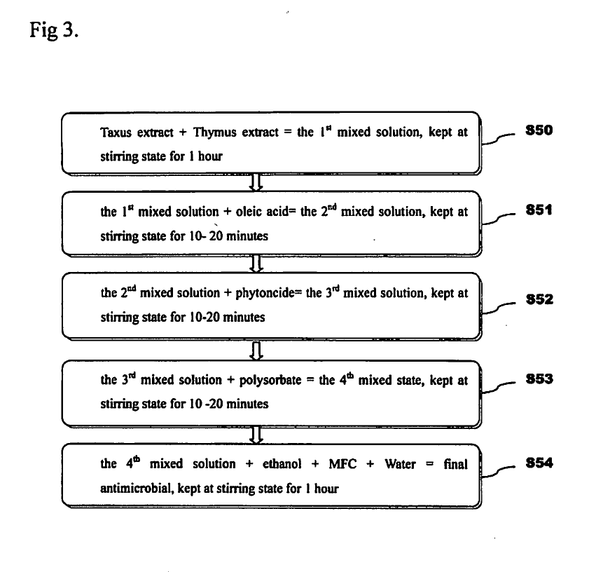

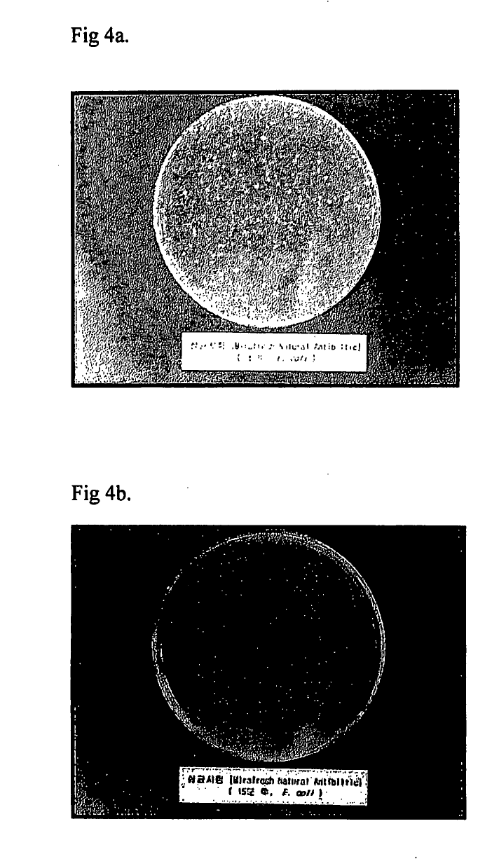

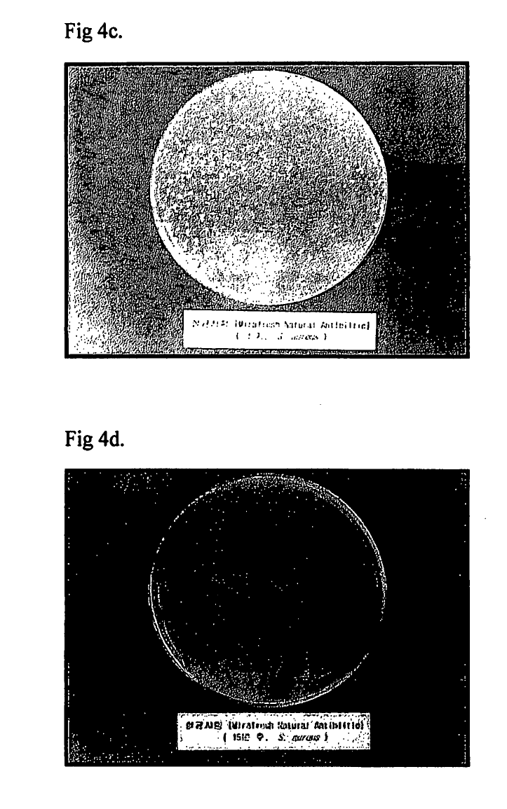

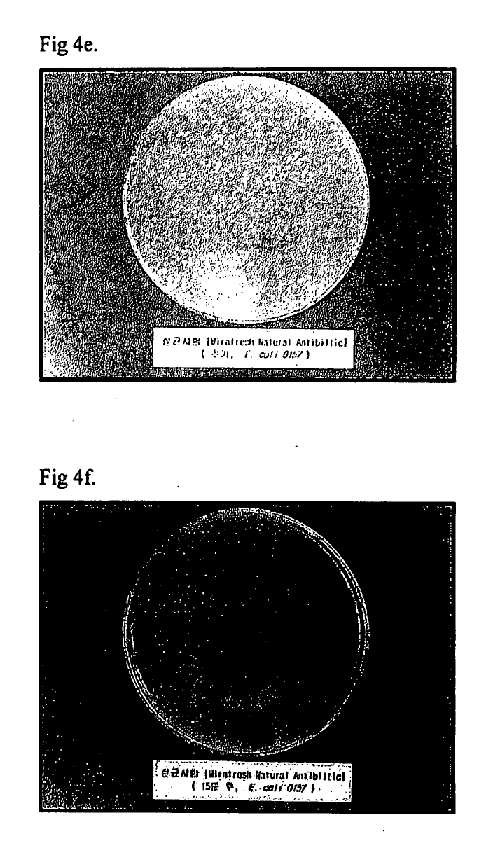

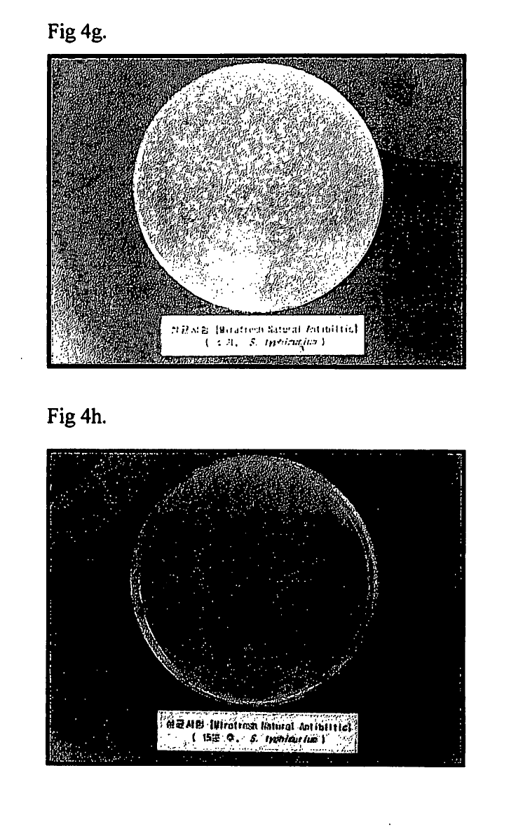

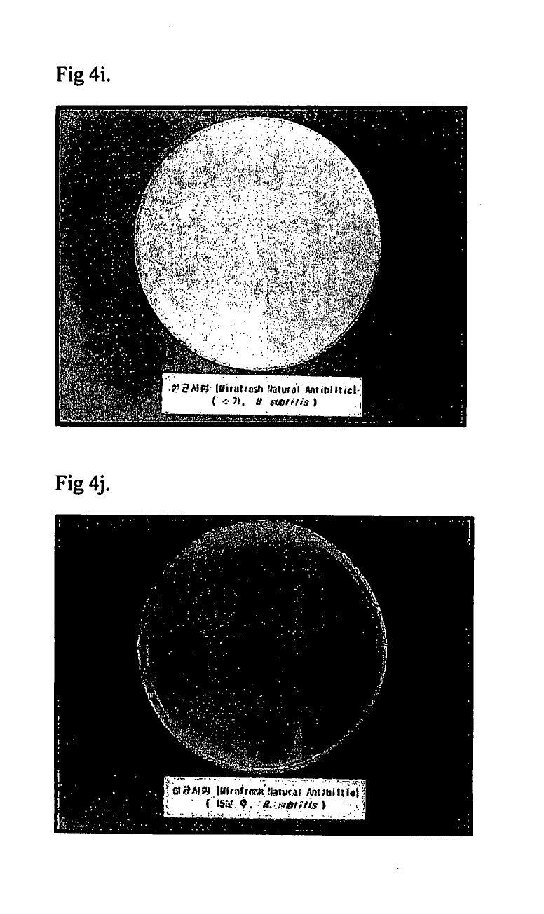

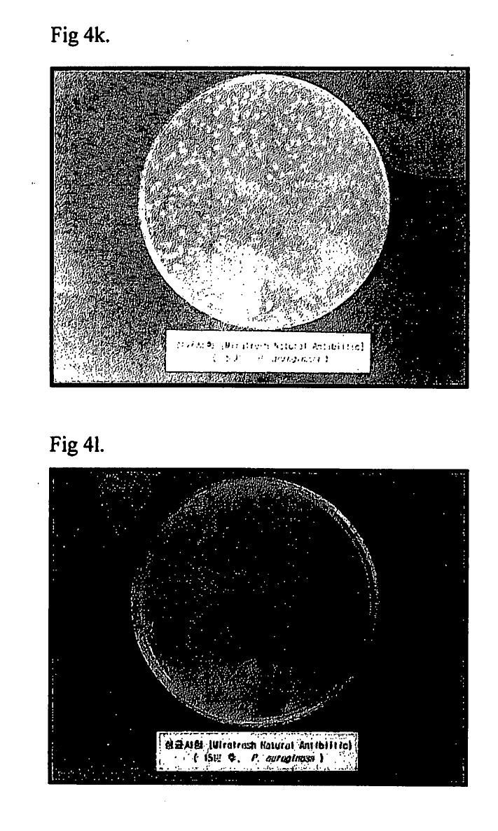

Explained below is the process in which the natural antimicrobial is manufactured using taxus extract and thymus extract as described above with reference to FIG. 3.

In other words, FIG. 3 is a flowchart that describes the process in which the composition substances of the natural antimicrobial according to the invention is manufactured.

Explained here is the process in which the composition substances of the natural antimicrobial according to the invention are manufactured with a basis on the ideal weight ratio shown in Table 1.

First, the 1^{st} mixed solution is created by mixing 50 g of Taxus extract, 50 g of Thymus extract, and 25 g of ethanol, and then this solution is kept at a constant stirring state for 1 hour (step S50).

In that state, the 1^{st} mixed solution described above is mixed with 37.5 g of Oleic Acid to create the 2^{nd} mixed solution, then the new solution is kept in constant stirring state for 10-20 minutes (step S51), then the 2^{nd} mixed solution is mixed with 5 g of phytoncide to create the 3^{rd} mixed solution, which is also then kept in a constant stirring state for 10-20 minutes (step S52).

The 3^{rd} mixed solution is mixed with 5 g of polysorbate to create the 4^{th} mixed solution, then is kept at a constant stirring state for 10-20 minutes (step S53), then the final natural antimicrobial is created by mixing the above 4^{th} mixed state with 25 g of ethanol, 20 g emulsification, and 782.5 g of water and keeping it in a constant stirring state for 1 hour.

On one side, the natural antimicrobial as described above is a germicide that uses emulsification, which is nano-fied natural antimicrobial substance that is manufactured using extracted substances from plant elements of Taxus and Thymus, as a delivery medium, and has no toxicity or corrosiveness against human body, animals, the environment, etc, and it is sprayed on surfaces of objects and human skin that may have been infected by microorganism to kill harmful germs as a disinfectant and to get rid of foul odor as a deodorant.

1. Experiment on the Disinfecting Capabilities of the Natural Antimicrobial

Described below are the results of the experiment on the disinfecting capabilities of the natural antimicrobial according to the invention.

The disinfecting capabilities of the natural antimicrobial were examined through an In Vitro Test of several of strains of germs.

1) Experimental Method

The experiment is to find out whether or not the experimented germ strains propagate, as well as the disinfecting capabilities of the antimicrobial by exposing the natural antimicrobial according to the invention to the germ strains for 15 minutes each.

2) The Experimented Germ Strains
Escherichia Coli: ATCC (AMERICAN TYPE CULTURE COLLECTION), 3 strains of clinical isolate type were experimented on.
Staphylococcus Aureus: ATCC, 3 strains of clinical isolate type were experimented on.
Escherichia coli 0157: ATCC, 3 strains of standard strains were experimented on.
Salmonella Typhiumnium: KCTC (Korea Collection For Type Culture), 3 strains of clinical isolate type were experimented on.
Bacillus Subtilis: KCTC, 1 strain of germ spore was experimented on.
Pseudomonas Aeruginosa: ATCC, 3 strains of clinical isolate types were experimented on.
Vibrio Parahaemolyticus: ATCC, 3 strains of clinical isolate types were experimented on.
Streptococcus Mutans: ATCC, 3 strains of clinical isolate types were used.

3) Initial Vaccination Germ Numbers
1˜9×10⁵ CFU/ml

4) Experiment Process

Each culture dishes containing the experimental strains are shaken in Brain Heart Infusion Broth, then these germs are diluted and then used in the experiment after it has been regulated so that the initial vaccination germ numbers is 1˜9×10⁵ CFU/ml.

20 ml of Experiment Solvent (sample undiluted solution) is put into the sterilized cap tube, then the initial vaccination germ numbers are regulated to dilute each of the cultivated germs, then the diminution rate of the initial germ numbers is inquired to estimate the number of germs 15 minutes after germ vaccination, using sterilized saline solution, which has the same amount as the experiment solvent, as a reference sample. However, in the first dilution step, the experiment is initiated after a neutralization process using D/E Neutralizing Broth (DIFCO).

In the case that the microbes propagate in the brain heart infusion broth, germ number was computed after multiplying it with number of dilutions, and in the case that the microbes didn't propagate, the germ number is labeled “less than 10” after multiplying it with number of dilutions that were done during the neutralization step.

Experiment Results

This experiment's results are as shown in the following Table 2.

TABLE 2

Number of Germs per 1 ml

| | | Initial | |
|---|---|---|---|
| Type of Spawn | Unit | Sample | After 15 Minutes |
| Escherichia coli | CFU/ml | 5.0*10⁵ | Less than 10 (over |
| | | | 99.9%) |
| Staphylococcus | CFU/ml | 6.2*10⁵ | Less than 10 (over |
| Aureus | | | 99.9%) |
| Escherichia coli | CFU/ml | 6.6*10⁵ | Less than 10 (over |
| 0157 | | | 99.9%) |
| Salmonella | CFU/ml | 5.1*10⁵ | Less than 10 (over |
| Typhiumrium | | | 99.9%) |
| Bacillus Subtilis | CFU/ml | 2.3*10⁵ | Less than 10 (over |
| | | | 99.9%) |
| Pseudomonas | CFU/ml | 3.3*10⁵ | Less than 10 (over |
| Aeruginosa | | | 99.9%) |
| Vibrio | CFU/ml | 4.3*10⁵ | Less than 10 (over |
| Parahaemolyticus | | | 99.9%) |
| Streptococcus | CFU/ml | 7.5*10⁵ | Less than 10 (over |
| Mutans | | | 99.9%) |

The rate of diminution after 15 minutes (over 99.9%) as shown above is calculated using the formula {(A−B)/A}×100. Here, A is the initial number of germs, and B is number of germs after a set time (i.e. 15 minutes).

As shown in the Table 2, it is revealed that the germs that were exposed to the natural antimicrobial according to the invention, Escherichia coli, staphylococcus aureus, Escherichia coli 157, salmonella typhiumrium, bacillus subtilis, pseudomonas aeruginosa, vibrio parahaemolyticus, and streptococcus mutans, all resulted in no propagation, and therefore it can be concluded that disinfection capabilities of the natural antimicrobial according to the current invention against general microbes as well as pathogenic microbes are very high.

FIGS. 4a to 4p show the result of the experiment conducted to see the disinfecting capabilities of the natural antimicrobial according to the invention on various germ strains.

As illustrated in FIG. 4a, FIG. 4c, FIG. 4e, FIG. 4g, FIG. 4i, FIG. 4k, FIG. 4m, and FIG. 4o, the cultivated germs Escherichia coli, staphylococcus aureus, Escherichia coli 157, salmonella typhiumrium, bacillus subtilis, pseudomonas aeruginosa, vibrio parahaemolyticus, and streptococcus mutans, after each being exposed to the natural antimicrobial for 15 minutes, not only stop propagating, over 99.9% of them are exterminated, as shown in FIG. 4b, FIG. 4d, FIG. 4f, FIG. 4h, FIG. 4j, FIG. 4l, FIG. 4n, and FIG. 4p.

Experiment on the Skin Stimulation of Natural Antimicrobial.

Explained below are the results of the experiment on the skin stimulation of natural antimicrobial according to the current invention. The experiment was conducted to find out the level of stimulation the natural antimicrobial according to the current invention has on the skin.

1) Experimental Method

6 male New Zealand White rabbits were applied the natural antimicrobial according to the invention, then death rates, general symptoms as well as weight change were studied for the next 72 hours, and the stimulation level of the affected areas at 24^{th} and 72^{nd} hour were assessed.

Experiment Materials
Species/Pedigree: New Zealand White Rabbit
Age and weight range during medication: six 3-4 months old male rabbit, weight range 2.0-3.0 kg
Quarantine and purification period: after the rabbits were obtained they were purified in the animal room for 6 days, only the animals judged healthy during this period were used in the experiment.
Environment conditions: the current experiment was conducted in a rabbit breeding room configured to temperature of 20±2□, relative humidity of 50±10%, ventilation frequency of 10˜12 times/hour, 12 hours of lighting, and illuminance of 200˜300 Lux.
Feeding and drink supply method: Lab animal rabbit feed (Purina Korea Co.) was given, and drinking water that had been disinfected by ultraviolet rays was given.
   3) Experiment's Construction

2 places in the nape of the neck of 6 of the healthy male rabbits were configured at about 2.5 cm×2.5 cm along the spines, upper part's 1 scratched place and 1 scratched place is configured as a treatment area where the experiment substances are treated onto, and lower part's 2 places are configured as non-treatment contrast area before carrying out the experiment. Scratched skin was made with the back of a microtome knife by damaging the outer skin, but it was made sure that the inner skin wasn't damaged and that there was no blood.

4) The Method of Application of the Natural Antimicrobial

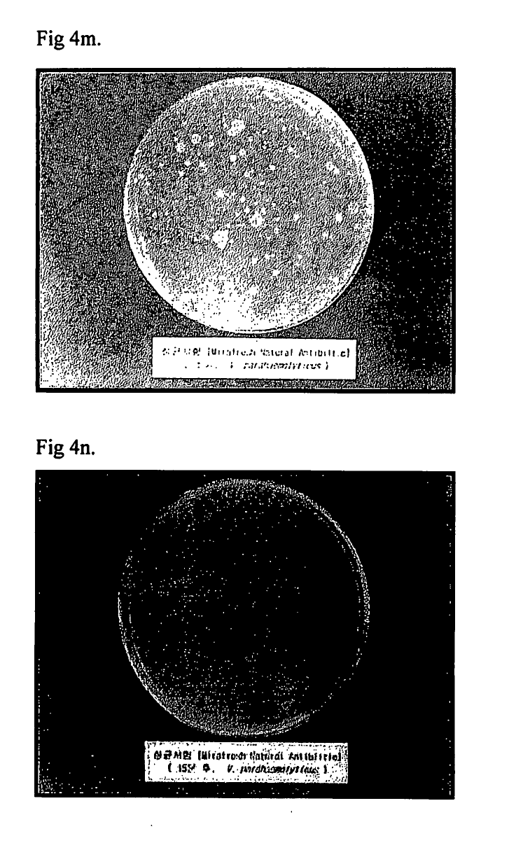

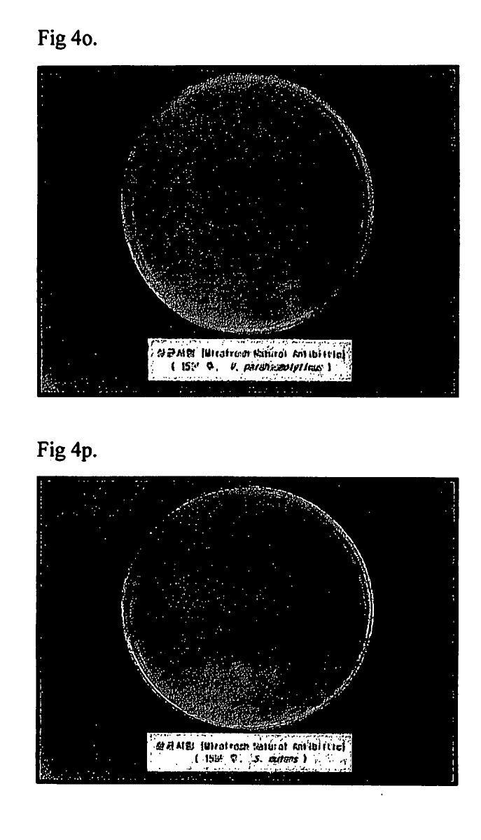

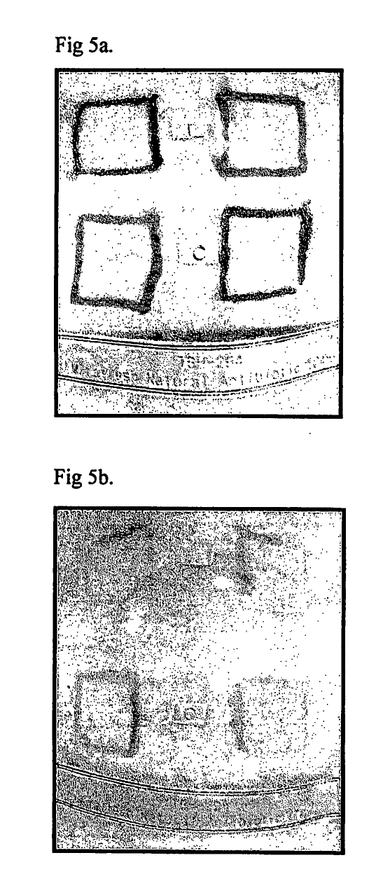

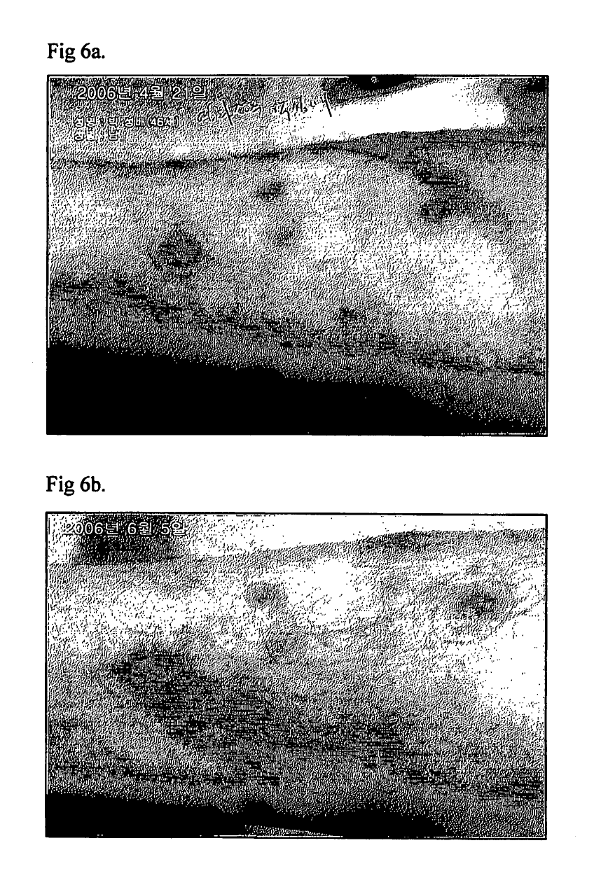

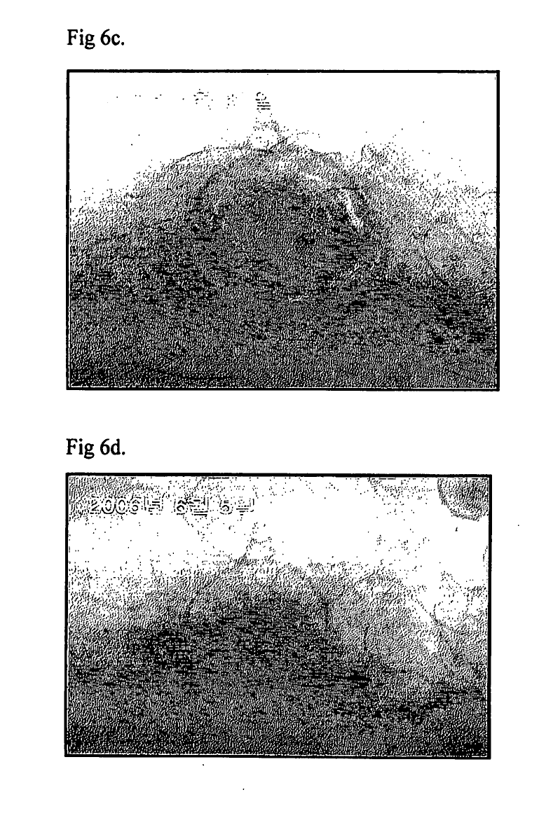

After the hair on the nape of the neck of the rabbit was removed 24 hours before the experiment, as shown in FIG. 5, 0.5 ml of the natural antimicrobial according to the current invention was applied to upper part's 1 scratched place and 1 non-scratched place, and sterilized saline solution was applied to the non-treatment contrast area with the same method.

After application, all the treated areas were secured with non-stimulating tapes and exposed for 24 hours.

Observation and Inspection Items
General symptoms observation: the existence general symptoms' changes, addiction symptoms, and deaths were observed for 72 hours after the application of the experiment substance.
Application area observation: existence of stimulating erythema, edema, and Incrustation are observed for 72 hours after the application of the experiment substance.
   6) Assessment of Skin Reaction and the Judgment of Stimulation Level

The assessment of skin reaction is based on Food and Drug Safety Office's notice number 2005-60's “Toxicity test standard of Medicinal supplies, etc (Oct. 21, 2005)”'s skin reaction assessment Table (refer to Table 3).

TABLE 3

| Erythema and Incrustation | | Edema formation | |
|---|---|---|---|
| | Assessment | Degree of | Assessment |
| Degree of symptom | Score | symptom | Score |
| No erythema | 0 | no edema | 0 |
| Very light erythema | 1 | very light | 1 |
| | | edema | |
| clear erythema | 2 | light edema | 2 |
| slightly excessive | 3 | normal | 3 |
| erythma | | edema | |
| Excessive erythema and | 4 | excessive | 4 |
| light Boil Scab | | edema | |

The judgment of level of stimulation based on the results is according to Draize's P.I.I. (Primary Irritation Index) calculation method (refer to Table 4).

TABLE 4

| Degree (P.I.I.) | Stimulated Area |
|---|---|
| 0.0-0.5 | No stimulation |
| 0.6-2.0 | weak stimulation |
| 2.1-5.0 | average stimulation |
| 5.1-8.0 | strong stimulation |

7) Experiment Results

General symptoms and death rates: in the case of all 6 rabbits there were no general symptoms or deaths observed.

Application area observation results: After observing the application areas of the 6 rabbits after 24 hours and 72 hours, as shown in FIG. 5b, there were no erythema, Incrustation, edema and other skin stimulations were observed on the treatment areas where the natural antimicrobial was applied, making all P.I.I. (Primary Irritation Index) scores “0.”

3. Experiment on the Antifungal Capabilities of the Natural Antimicrobial

Explained below are the results of the experiment on the inspection of the antifungal capabilities of the natural antimicrobial according to the current invention.

The natural antimicrobial's skin ailment healing capabilities were tested using Rubrum as the skin ailment germ in a Halo Test.

1) Experiment Method

After making Spore solution out of Trichophyton Rubrum stored in the four sided Sabouraud Dextrose Agar, 200 ul of spore solution is rod cultured on the sabouraud dextrose agar, then a 8 mm sterilized disk is placed on the center of Sabouraud Dextrose Agar and 50 ul of the natural antimicrobial according to the current invention is applied on the disk. After cultivating at 25±1° for 7 days, the diameter of the inhibition zone is estimated.

2) Germ Strain being Experimented
Trichophyton Rubrum: KCTC (Korea Collection For Type Culture)

3) Experiment Result

The experiment result is as shown below in Table 5

TABLE 5

| Experimented | | Width of the |
|---|---|---|
| Strain | Unit | Inhibition Zone |
| trichophyton | mm | 1.9 |
| Rubrum | | |

As shown above in Table 5, an Inhibition Zone with width of 1.9 mm was formed as a result of a Halo test, which was conducted to find out the antifungal effects of natural antimicrobial on dermatophytes.

Next, FIGS. 6a to 6i show an exemplified process of the healing process of the human skin ailment area after the natural antimicrobial according to the invention is applied.

As illustrated in FIG. 6a to FIG. 6d, a patient with a severe case of Psoriasis on the leg saw the disease fully cured after about 5 weeks of periodical application of the natural antimicrobial according to the current invention.

As illustrated in FIGS. 6e and 6f, a patient who was suffering from eczema inside the earflap was cured after about 4 weeks of periodical application of the natural antimicrobial according to the current invention.

As illustrated in FIG. 6g, a patient who was suffering from eczema around the ear was also cured after about 4 weeks of periodical application of the natural antimicrobial according to the current invention.

As illustrated in FIGS. 6h and 6i, a patient suffering from leg ailments caused by athlete's foot germs, foot odor, spots, blisters and itching was rid of leg ailments, spots, and blisters after 3 weeks of periodical application of the natural antimicrobial according to the current invention, and foot odor and itching were almost fully cured as well.

Furthermore explained is the disinfection of molds using the natural antimicrobial according to the current invention, with reference to the attached illustrations.

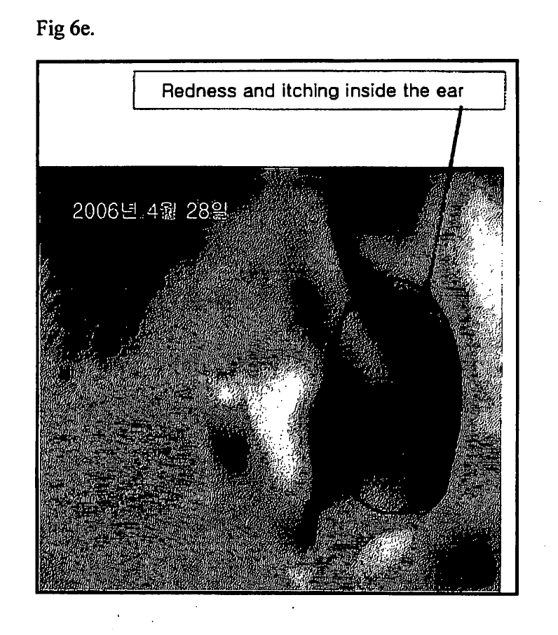

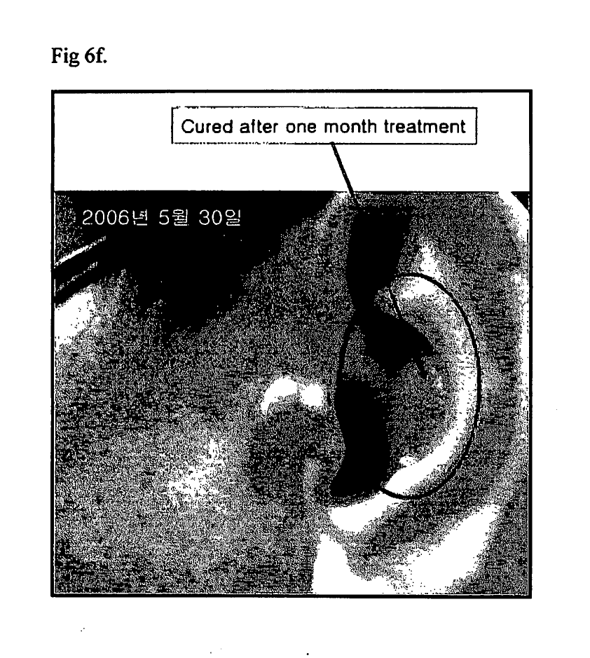

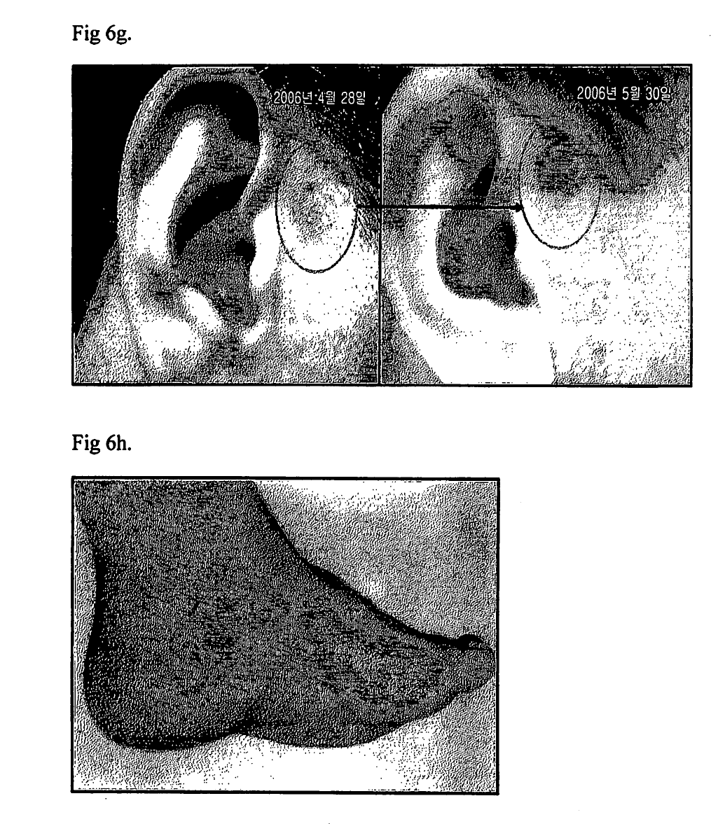

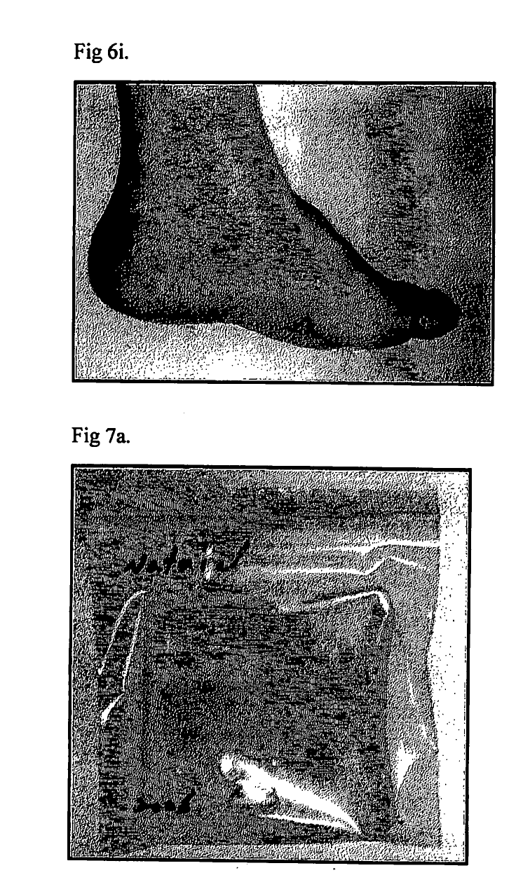

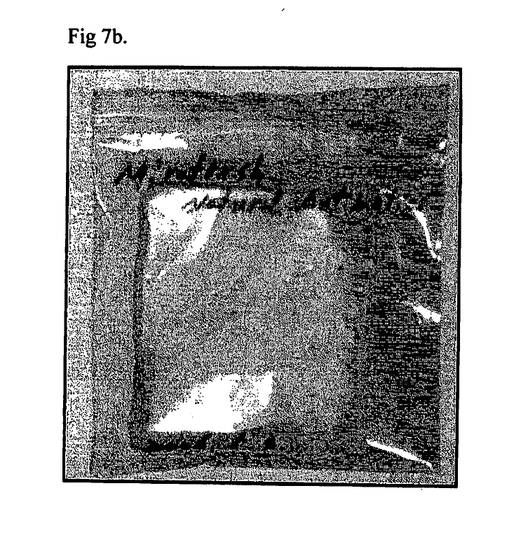

In other words, FIGS. 7a and 7b are exemplifying pictures that compare bread that has been applied the natural antimicrobial and bread that has not been applied the natural antimicrobial.

After sealing up bread applied with the natural antimicrobial and bread not applied with the natural antimicrobial in a 20° condition for a week, it has been shown in FIG. 7a that bread not applied with natural antimicrobial had extreme propagation of molds, while bread applied with natural antimicrobial in FIG. 7b didn't have any propagation of molds.

The natural antimicrobial according to the current invention has antimicrobial and exterminating functions against a wide range of microbes, maleficent pathogenic germs and bacteria such as athlete's foot germs, fungus, and molds, allowing for its use on a wide range of objects and environments such as clothing, furniture, shoes, and kitchen utensils where these microbes, pathogens, and bacteria can easily propagate.

Also, the natural antimicrobial according to the current invention's main ingredients are all natural plant components that are harmless to the human body as well as animals, making it possible to use it to treat skin ailments caused by various microbes, pathogens, and bacteria that occur on the human body and animals, as well as on objects that frequently makes contacts with the human body, namely as disinfectant wipes, nursing bottle cleaner, and female vaginal cleaners.

Explained and illustrated above were specific executions of the current invention, it is self-evident that the current invention may have various changed executions based on the industry in question. These changes should not be understood individually from the current invention's technical idea or prospect, but should be understood as being included inside the range of the claims attached to the current invention.

According to the invention as described above, it is possible to produce all natural antimicrobial capable of repressing and exterminating the propagation of various microbes, pathogens, and bacterium using natural extracts from Taxus and Thymus, and therefore capable of being used on a variety of objects and environments such as clothing, furniture, and kitchen utensils where various microbes, pathogens and bacterium can propagate, and, because the main ingredient is extract from all natural plants, it can be used to treat a variety of skin ailments breaking out on the human body and animals that are caused by various microbes, pathogens, and bacteria without any harmful side effects.

While the invention has been shown and described with reference to a certain preferred embodiments thereof, it will be understood by those skilled in the art that various changes in form and details may be made therein without departing from the spirit and scope of the invention as defined by the appended claims.

I claim:

1. A natural antimicrobial comprising:
a taxus extract from Taxus and Thymus extract.

2. The natural antimicrobial according to claim 1, further comprising oleic acid, ethanol, deionized water, and emulsification.

3. The natural antimicrobial according to claim 2, further comprising polysorbate and phytoncide.

4. The natural antimicrobial according to claim 3, comprising weight ratio of 2-7 Weight % Taxus extract, 2-7 Weight % Thymus extract, 2-5 Weight % Oleic Acid, 0.1-1.0 Weight % polysorbate, 0.1-1.0 Weight % phytoncide, 3-8 Weight % ethanol, 75-80 Weight % water, and 1-3 Weight % nano emulsion.

5. The natural antimicrobial according to claim 1, wherein the Taxus extract further comprises Taxine of alkaloid type, Taxinine A, B, H, K, L, and Taxol.

6. The natural antimicrobial according to claim 5, wherein Taxus extract further comprise Ponasterone A, Sciadopitysin, Taxusin, Ecdysterone.

7. The natural antimicrobial according to claim 6, wherein Taxus extract comprises weight ratio of 12.87-15.73 Weight % Taxine, 5.39-6.58 Weight % Ponasterone A, 3.90-4.78 Weight % Sciadopitysin, 12.33-15.07 Weight % Taxusin, 11.04-16/56 Weight % Taxinine A, B, H, K, L, 4.04-6.06 Weight % Ecdysterone, 38.03-46.47 Weight % Taxol, and 0.70-1.06 Weight % other substances.

8. The natural antimicrobial according to claim 1, wherein Thymus extract further comprises Thymol and Ketoconazole.

9. The natural antimicrobial according to claim 8, wherein Thymus extract further comprises Cavacrol, γ-Terpinene, Borneol, \acute{α}(alpha)-Terpineol, \acute{α}(alpha)-Pinene, and P-Cymene.

10. The natural antimicrobial according to claim 9, wherein Thymus extract comprises weight ratio of 31.41-38.39 Weight % Thymol, 4.77-5.83 Weight % Carvacrol, 7.2-8.8 Weight % γ-Terpinene, 4.5-5.5 Weight % Borneol, 30.18-36.88 Weight % Ketoconazole, 1.36-2.04 Weight % P-Cymene, 4.0-6.0 Weight % \acute{α}(alpha)-Terpineol, 4.55-6.83 Weight % \acute{α}(alpha)-Pinene, and 0.46-0.7 Weight % Other substances.

11. A method of manufacturing a natural antimicrobial, the method comprising:
creating a 1^{st} mixed solution by mixing Taxus extract, Thymus extract and ethanol;
creating a 2^{nd} mixed solution by mixing oleic acid with the 1^{st} mixed solution;
creating a 3^{rd} mixed solution by mixing phytoncide with the 2^{nd} mixed solution; and
creating a 4^{th} mixed solution by mixing polysorbate with the 3^{rd} mixed solution,
wherein the natural antimicrobial is completed by mixing ethanol, emulsification and water with the 4^{th} mixed solution.

12. The method of manufacturing the natural antimicrobial according to claim 11, wherein Taxus extract is obtained by:
breaking Taxus components into pieces, which are then rid of impurities by washing with water;
ridding the Taxus components of remaining moisture;
grinding the broken Taxus components into powder, mixing with ethanol solvent, and then stirring;
filtering the mixed solution of Taxus powder and ethanol solvent, and separating liquid substance from solid substance;
mixing the liquid substance with a solvent to eliminate impurities; and
completing extract Taxus by removing the mixed solvent and dehydrating the liquid substance.

13. The method of manufacturing the natural antimicrobial according to claim 12, wherein the step of grinding the broken Taxus components further comprises grinding dehydrated taxus components into powder and then mixing with ethanol solvent and stirred, mixing at a slow speed a weight ratio of taxus powder to ethanol

14. The method of claim 12, wherein the weight ratio is 1:100.

15. The method of manufacturing the natural antimicrobial according to claim 12, wherein the step of mixing the liquid substance with a solvent to remove impurities further comprises:
mixing at a slow speed a solvent, dicholomethane and purifying water, at a weight ratio of 1:1:1; and
ridding the mixed substance of impurities, and leaving to precipitate.

16. The method of manufacturing the natural antimicrobial according to claim 12, wherein the step of completing Taxus extract comprises:
vaporizing and removing the solvent to obtain a concentrated liquid; and
completing the Taxus extract by heating and dehydrating the concentrated liquid.

17. The method of manufacturing the natural antimicrobial according to claim 11, wherein the step of completing Taxus extract comprises:
breaking the thymus components into pieces, and washing by water to remove impurities;
removing remaining moisture in the thymus components;
grinding the thymus pieces into power and mixing with purifying water, then mixing by a mixer;
purifying and filtering the thymus powder mixture to separate the liquefied component from the solid components;
mixing the liquefied component with a solvent to remove impurities; and
completing the thymus extract by removing the mixed solvent and moisture.

18. The method of manufacturing the natural antimicrobial according to claim 16, wherein, in the step of grinding the thymus pieces into power, mixing is performed at a slow speed with a weight ratio of thymus powder to purifying water of 1:10,
and wherein in the step of mixing the liquefied component with a solvent, a weight ratio liquefied component and ethanol solvent is 1:1.
